(19) United States
(12) Patent Application Publication
(10) Pub. No.: US 2013/0079571 A1
(43) Pub. Date: Mar. 28, 2013

(54) HYDROCARBON CONVERSION METHOD AND APPARATUS

(71) Applicant: Laurence O. Stine, Western Spring, IL (US)

(72) Inventor: Laurence O. Stine, Western Spring, IL (US)

(73) Assignee: UOP, LLC., Des Plaines, IL (US)

(21) Appl. No.: 13/244,006

(22) Filed: Sep. 23, 2011

(51) Int. Cl. C07C 4/06; B01J 8/08

(52) U.S. Cl. 585/310; 422/630; 422/187

(57) ABSTRACT

One exemplary embodiment can be a hydrocarbon conversion method. Generally, the method includes providing a hydrocarbon stream having one or more C10-C14 hydrocarbons to a hydroprocessing zone and a donor solvent stream at least partially obtained from the hydroprocessing zone to a slurry hydrocracking zone. The hydroprocessing zone may have a vessel containing an internal riser. Usually, a hydroprocessing catalyst circulates within the vessel by at least partially rising within the internal riser.

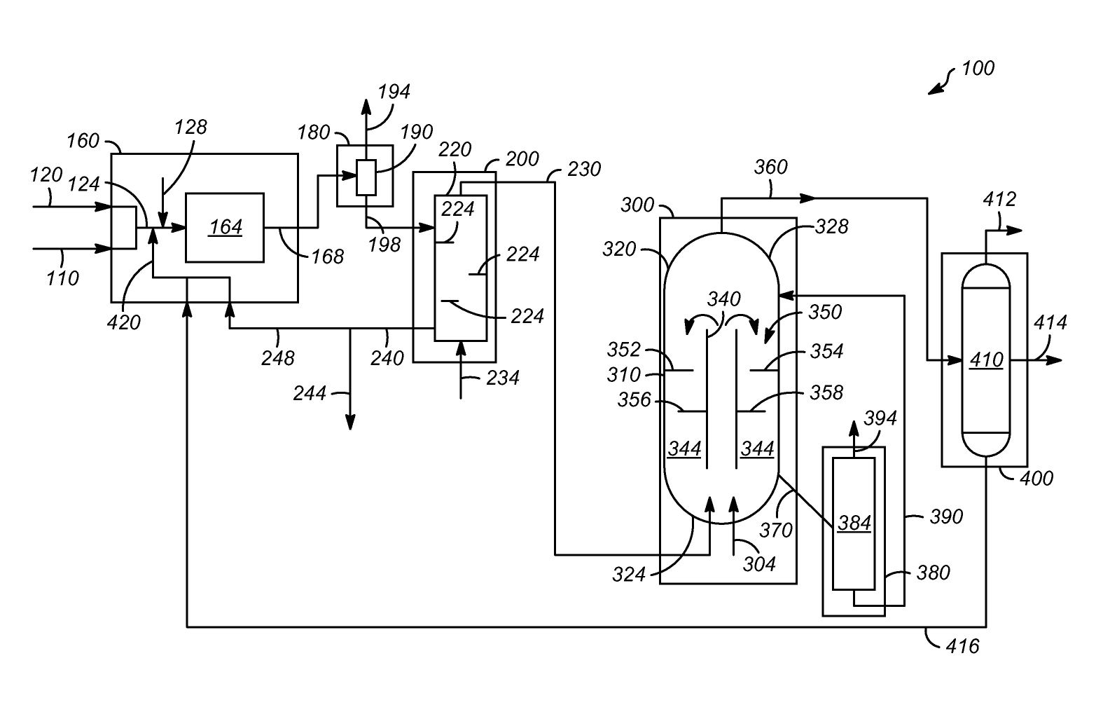

### FIELD OF THE INVENTION

This invention generally relates to a hydrocarbon conversion method and apparatus.

### DESCRIPTION OF THE RELATED ART

Hydroprocessing heavier hydrocarbons can utilize a variety of processes, such as slurry hydrocracking. Often, it is desirous to improve the efficiency of such a process because the feed has a large amount of condensed and heterogeneous cyclic compounds. However, the effectiveness of such systems can be reduced due to low catalyst diffusion rates in a liquid phase. Thus, there is a desire to provide additional units that can, in turn, provide a solvent to improve diffusion and provide additional hydrogen to increase reactivity.

### SUMMARY OF THE INVENTION

One exemplary embodiment can be a hydrocarbon conversion method. Generally, the method includes providing a hydrocarbon stream having one or more C10-C14 hydrocarbons to a hydroprocessing zone and a donor solvent stream at least partially obtained from the hydroprocessing zone to a slurry hydrocracking zone. The hydroprocessing zone may have a vessel containing an internal riser. Usually, a hydroprocessing catalyst circulates within the vessel by at least partially rising within the internal riser.

Another exemplary embodiment can be a hydrocarbon conversion method. Usually, the method includes providing a hydrocarbon stream having one or more C10-C14 hydrocarbons to a hydroprocessing zone, a product stream from the hydroprocessing zone to a fractionation zone for obtaining a donor solvent stream, and the donor solvent stream at least partially obtained from the hydroprocessing zone to a slurry hydrocracking zone. Typically, the hydroprocessing zone has a vessel containing an internal riser. Usually, a hydroprocessing catalyst circulates within the vessel by at least partially rising within the internal riser.

A further exemplary embodiment may be a hydrocarbon conversion apparatus. The hydrocarbon conversion apparatus may include a hydroprocessing zone and a slurry hydrocracking zone. Usually, the hydroprocessing zone includes a vessel containing an internal riser, which can be wholly contained by a shell of the vessel. Typically, the slurry hydrocracking zone includes a slurry hydrocracking reactor in communication with the hydroprocessing zone to receive a hydrocarbon stream at least partially obtained from the hydroprocessing zone.

The embodiments provided herein can reduce the viscosity of the liquid utilized in the slurry hydrocracking zone. Particularly, a donor solvent provided from a hydroprocessing zone can be provided to the slurry hydrocracking zone. The donor solvent can be a multi-ring compound with excess hydrogen that can be shifted to a hydrogen deficient molecule, such as an alkene derived from the cracking conditions. The donor solvent can include at least one of decahydronaphthalene and tetrahydronaphthalene. Alternatively, tricyclic and heavier compounds may also be utilized. Thus, the viscosity of the liquid may be lowered improving diffusion rates of the catalyst, and additional hydrogen can be provided via the donor solvent. Often, hydrogen obtained from a donor solvent is more reactive than molecular hydrogen. Alternatively, a stream including other hydrocarbons, such as heavier hydrocarbons, e.g., a vacuum gas oil, may be provided instead of a donor solvent.

### DEFINITIONS

As used herein, the term “stream” can be a stream including various hydrocarbon molecules, such as straight-chain, branched, or cyclic alkanes, alkenes, alkadienes, and alkynes, and optionally other substances, such as gases, e.g., hydrogen, or impurities, such as heavy metals, and sulfur and nitrogen compounds. The stream can also include aromatic and non-aromatic hydrocarbons. Moreover, the hydrocarbon molecules may be abbreviated C1, C2, C3 . . . Cn where “n” represents the number of carbon atoms in the one or more hydrocarbon molecules.

As used herein, the term “zone” can refer to an area including one or more equipment items and/or one or more sub-zones. Equipment items can include one or more reactors or reactor vessels, heaters, exchangers, pipes, pumps, compressors, and controllers. Additionally, an equipment item, such as a reactor, dryer, or vessel, can further include one or more zones or sub-zones.

As used herein, the term “rich” can mean an amount of at least generally about 50%, and preferably about 70%, by mole, of a compound or class of compounds in a stream.

As used herein, the term “substantially” can mean an amount of generally at least about 80%, preferably about 90%, and optimally about 99%, by weight, of a compound or class of compounds in a stream.

As used herein, the term “feed” as provided to a slurry hydrocracking zone can include of one or more hydrocarbons and optionally hydrogen, a slurry hydrocracking catalyst, and/or one or more recycled materials. In some instances, the one or more hydrocarbons may be referred to as a feed separate from the hydrogen, slurry hydrocracking catalyst, and/or recycled materials.

As used herein, the term “hydroprocessing” can include hydrotreating and/or hydrocracking.

As depicted, process flow lines in the figures can be referred to interchangeably as, e.g., lines, pipes, feeds, products, or streams.

### BRIEF DESCRIPTION OF THE DRAWING

The FIGURE is a schematic depiction of an exemplary hydrocarbon conversion apparatus.

### DETAILED DESCRIPTION

Referring to the FIGURE, a hydrocarbon conversion apparatus 100 can include a slurry hydrocracking zone 160, a flash zone 180, a stripping zone 200, a hydroprocessing zone 300, a regeneration zone 380, and a fractionation zone 400. Generally, a feed 110 includes at least about 10%, by weight, one or more hydrocarbons boiling above about 500° C. However, any suitable feed 110 or combination feeds can be provided, such as a vacuum gas oil, a vacuum residue, or a fluidized catalytic cracking gas oil boiling above about 400° C., above about 425° C., or even above about 510° C. Typically, the feed 110 provided to the slurry hydrocracking zone 160 may have, e.g., about 90%, by weight, boiling above a temperature of at least about 300° C. at an atmospheric equivalent boiling point as calculated from observed boiling temperature and distillation pressure, as determined by ASTM D1160-06. Such a feed 110 can have an API gravity of no more than about 20°, and typically no more than about 10°. The feed 110 can be combined with a hydrogen stream 120 to form a combined feed 124. Furthermore, a combined stream 420 may be added to the combined feed 124, as hereinafter described.

The hydrogen stream 120 can include any suitable amount of hydrogen effective to promote a slurry hydrocracking reaction, such as at least about 30%, by volume, preferably at least about 80%, by volume, hydrogen. Generally, the hydrogen stream 120 can include recycled and/or make-up hydrogen, and as such can include other light hydrocarbon molecules, such as methane, ethane, and ethene, and inert gases such as nitrogen.

The slurry hydrocracking zone 160 can include a slurry hydrocracking reactor 164. Generally, a catalyst stream 128 is provided to form a mixture that is processed in the slurry hydrocarbon reactor 164. Generally, the slurry hydrocarbon cracking catalyst can include particles having a mean particle diameter of about 2-about 100 microns.

Exemplary catalyst compounds can include a catalytically effective amount of one or more compounds having iron. Particularly, the one or more compounds can include at least one of an iron oxide, an iron sulfate, and an iron carbonate. Other forms of iron can include at least one of an iron sulfide, a pyrrhotite, and a pyrite. What is more, the catalyst can contain materials other than an iron, such as at least one of molybdenum, nickel, and manganese, and/or a salt, an oxide, and/or a mineral thereof.

Preferably, the one or more compounds includes an iron sulfate, and more preferably, at least one of an iron sulfate monohydrate and an iron sulfate heptahydrate. Oxidic iron-containing compounds obtained from sources such as a limonite, a laterite, a wrought iron, a clay, a magnetite, a hematite, a gibbsite, or a Kisch iron can also be used. One particularly desired material is ferrous sulfate, which can either be a monohydrate or a heptahydrate.

Desirably, one or more catalyst particles can include about 2-about 45%, by weight, iron oxide and about 20-about 90%, by weight, alumina. In one exemplary embodiment, iron-containing bauxite is a preferred material having these proportions. Bauxite can have about 10-about 40%, by weight, iron oxide (Fe₂O₃), and about 54-about 84%, by weight, alumina and may have about 10-about 35%, by weight, iron oxide and about 55-about 80%, by weight, alumina. Bauxite also may include silica (SiO₂) and titania (TiO₂) in amounts of usually no more than about 10%, by weight, and typically in amounts of no more than about 6%, by weight. Volatiles such as water and carbon dioxide may also be present, but the foregoing weight proportions exclude such volatiles. Iron oxide is also present in bauxite in a hydrated form, but again the foregoing proportions exclude water in the hydrated composition.

In another exemplary embodiment, it may be desirable for the catalyst to be supported. Such a supported catalyst can be relatively resilient and maintain its particle size after being processed through the slurry hydrocracking zone 160. As a consequence, such a catalyst can include a support of alumina, silica, titania, one or more aluminosilicates, magnesia, bauxite, coal and/or petroleum coke. Such a supported catalyst can include a catalytically active metal, such as at least one of iron, molybdenum, nickel, and vanadium, as well as sulfides of one or more of these metals. Generally, the catalyst can have about 0.01-about 30%, by weight, of the catalytic active metal based on the total weight of the catalyst.

Generally, the slurry hydrocracking reactor 164 can operate either in up-flow or down-flow. One exemplary reactor can be a tubular reactor through which the feed, catalyst, and gas pass upward. Generally, the temperature can be about 400-about 500° C., preferably about 440-about 465° C., and a pressure of about 3-about 24 MPa, preferably about 10-about 18 MPa. The liquid hourly space velocity is typically below about 4 hr⁻¹. Exemplary slurry hydrocracking zones and catalyst are disclosed in, e.g., U.S. application Ser. No. 12/813,468 filed 10 Jun. 2010.

An effluent 168 from the slurry hydrocracking zone 160 can be provided or passed to a flash zone 180 including a flash drum 190. Generally, the flash zone 180 is provided to separate lighter materials from the effluent 168, such as hydrogen and light gases, typically methane, ethane, and ethene. Optionally, these gases in a line 194 can be routed back to the slurry hydrocracking zone 160. A line 198 can route heavier hydrocarbons, such as vacuum gas oil and pitch, to the stripping zone 200. The stripping zone 200 may be adapted to receive at least a portion of the effluent from the slurry hydrocracking zone 160 and can include a stripper 220 having one or more baffles 224. Generally, the stripper 220 may receive a hydrogen stream 234, which can include any suitable amount of hydrogen. Exemplary amounts of hydrogen may include at least about 30%, by volume, preferably at least about 80%, by volume, nitrogen. Generally, the hydrogen stream 120 can include recycled and/or make-up hydrogen, and as such can include other light hydrocarbon molecules, such as methane, ethane, and ethene, and inert gases such as nitrogen.

A bottom stream 240 can exit the stripper 220 and be split with a catalyst purge stream 244 removing excess and spent catalyst, and a recycle stream 248 including heavy hydrocarbons and some catalyst returned to the slurry hydrocracking zone 160. The recycle stream 248 can be combined with a donor solvent stream 416, as hereinafter described, to form a combined stream 420 before being added to the combined feed 124 and prior to combination with the catalyst stream 128. Although the streams 110, 120, 248, and 416 are depicted as being combined outside the slurry hydrocracking reactor 164, it should be understood that one or more of these streams 110, 120, 248, and 416 may be provided directly to the slurry hydrocracking reactor 164. A stream 230, typically from the top of the stripper 220, can include one or more C10-C14 hydrocarbons, often a kerosene and/or diesel cut, that can be provided to the hydroprocessing zone 300. Usually, the stream 230 is in a gas phase.

The hydroprocessing zone 300 can include a vessel 310. Usually, the vessel 310 can include a shell 320, a base 324, a top 328, and internal riser 340. Generally, the internal riser 340 is wholly contained by the shell 320 of the vessel 310. Optionally, at least about 90%, or even about 99%, of a length of the internal riser 340 is contained by the shell 320 of the vessel 310. The shell 320 and the internal riser 340 may form an annulus 344.

Typically, the vessel 310 can be operated at any suitable temperature and pressure, such as a temperature of about 250-about 650° C., preferably about 250-about 500° C., and a pressure of about 1,300-about 140,000 kPa, preferably about 3,000-about 35,000 kPa, and optimally about 3,500-about 14,000 kPa. Usually, the catalyst is provided in a ratio of about 1:1-about 100:1, preferably about 1:1-about 20:1, by weight, of catalyst to the stream 230. Any suitable liquid hourly space velocity, such as about 0.1-about 10 hr⁻¹, preferably about 0.5-about 3 hr⁻¹, may be utilized. Typically, it is preferred that the catalyst is free of any sulfide containing gases, such as hydrogen sulfide or other contaminants. A ratio of hydrogen to hydrocarbon feed can be about 80-about 3,600 m³/m³, preferably about 170-about 1,800 m³/m³. Typically, the vessel 310 can receive a hydrogen stream 304 to facilitate the hydrocracking reactions. Any suitable amount of hydrogen effective to promote a hydrocarbon reaction may be utilized, such as at least about 30%, by volume, preferably at least about 80%, by volume, hydrogen. Generally, the hydrogen stream 304 can include recycled and/or make-up hydrogen, and as such can include other light hydrocarbon molecules, such as methane, ethane, and ethene, and inert gases such as nitrogen.

Typically, hydroprocessing catalyst circulates within the vessel 310 by at least partially rising within the internal riser 340 and dropping in the annulus 344. The passage of the catalyst can be slowed by a plurality of pans, namely a first pan 352, a second pan 354, a third pan 356, and a fourth pan 358.

Any suitable hydroprocessing catalyst can be utilized. The catalyst may be an inorganic oxide material, which can include porous or non-porous catalyst materials of at least one of a silica, an alumina, a titania, a zirconia, a carbon, a silicon carbide, a silica-alumina, an oil sand, a diatomaceous earth, a shale, a clay, a magnesium, an activated carbon, a fused-carbon from heavy oil or coal, and a molecular sieve. A silica alumina may be amorphous or crystalline and include silicon oxide structural units. Optionally, the catalyst can include a metal deposited on the inorganic oxide material. A suitable metal deposited on the support may include at least one element from a group 6 and groups 8-10 of the periodic table. The catalyst may include one or more metals of chromium, molybdenum, zirconium, zinc, tungsten, iron, ruthenium, osmium, cobalt, rhodium, iridium, nickel, palladium, and platinum, and preferably may include platinum or palladium. The metal component of the group 6 can be in an amount of about 1-about 20%, by weight; the iron-group metal component of groups 8-10 may be in an amount of about 0.2-about 10%, by weight; and the noble metal of groups 8-10 can be in an amount of about 0.1-about 5%, by weight, based on the total weight of the catalyst. It is further contemplated that the catalyst may also include at least one of cesium, francium, lithium, potassium, rubidium, sodium, copper, gold, silver, cadmium, mercury and zinc. The catalyst may be formed into spheres and spray-dried.

Alternatively, the catalyst includes two components or catalysts, namely a first component or catalyst such as an active amorphous clay and/or a high activity crystalline molecular sieve, and a second component or catalyst such as a medium or smaller pore zeolite. Such a catalyst mixture is disclosed in, e.g., U.S. Pat. No. 7,312,370 B2. Still yet another embodiment can be a slurry catalyst composition, which may include a catalytically effective amount of one or more compounds having iron. Particularly, the one or more compounds can include at least one of an iron oxide, an iron sulfate, and an iron carbonate. Other forms of iron can include at least one of an iron sulfide, a pyrrhotite, and a pyrite. What is more, the catalyst can contain materials other than an iron, such as at least one of molybdenum, nickel, and manganese, and/or a salt, an oxide, and/or a mineral thereof.

Generally, the catalyst or at least a portion can be no more than about 1,000 microns, preferably may be no more than about 500 microns, even preferably no more than about 100 microns, and optimally no more than about 50 microns, in mean particle diameter, to facilitate reactions and increase the overall surface area of the catalyst. In one exemplary embodiment, the catalyst may have a mean particle diameter of about 50-about 100 microns.

A first catalyst line 370 can provide spent catalyst to the regeneration zone 380, which may be adapted to receive a spent catalyst and can include a regeneration vessel 384. The regeneration vessel 384 may operate at any suitable conditions such as a pressure of about 100-about 6,900 kPa and a temperature of about 450-about 550° C.

The regeneration vessel 384 can receive oxygen to regenerate the catalyst and provide a flue gas stream 394 and return regenerated catalyst through a second catalyst line 390 proximate to the top 328 of the hydrocracking vessel 310. It should be known that other equipment can also be used in conjunction with the vessel 310 and the regeneration vessel 384, such as one or more lock hoppers, cyclone separators, steam strippers, and various valves. Exemplary hydrocracking and regeneration vessels are disclosed in, e.g., U.S. application Ser. No. 13/007,583 filed 14 Jan. 2011 and U.S. application Ser. No. 13/051,854 filed 18 Mar. 2011.

A product stream 360 including hydrogen and one or more C1-C14 hydrocarbons can exit the hydroprocessing zone 300 and be provided to the fractionation zone 400 adapted to receive the product stream 360. The fractionation zone 400 can include a fractionation column 410 providing a stream 412 including one or more C4⁻ hydrocarbons, a stream 414 including one or more C5-C9 hydrocarbons, and a donor solvent stream 416 at least partially obtained from the hydroprocessing zone 300. The donor solvent stream 416 may be separated from the product stream 360 and can include any suitable hydrocarbon that can provide hydrogen to facilitate reactions. Exemplary hydrocarbons can include at least one of decahydronaphthalene and tetrahydronaphthalene. The donor solvent stream 416 can be provided to the slurry hydrocracking zone 160 and be optionally combined with the recycle stream 248, as described above. Alternatively, a hydrocarbon stream can be provided that includes, e.g., heavier hydrocarbons, such as a vacuum gas oil, instead of donor solvents.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing, all temperatures are set forth in degrees Celsius and, all parts and percentages are by weight, unless otherwise indicated.

From the foregoing description, one skilled in the art can easily ascertain the essential characteristics of this invention and, without departing from the spirit and scope thereof, can make various changes and modifications of the invention to adapt it to various usages and conditions.

1. A hydrocarbon conversion method, comprising:
A) providing a hydrocarbon stream comprising one or more C10-C14 hydrocarbons to a hydroprocessing zone comprising a vessel containing an internal riser; wherein a hydroprocessing catalyst circulates within the vessel by at least partially rising within the internal riser; and
B) providing a donor solvent stream at least partially obtained from the hydroprocessing zone to a slurry hydrocracking zone.

2. The method according to claim 1, wherein the donor solvent stream comprises at least one of decahydronaphthalene and tetrahydronaphthalene.

3. The method according to claim 1, further comprising providing a feed comprising at least about 10%, by weight, one or more hydrocarbons boiling above about 500° C. to the slurry hydrocracking zone.

4. The method according to claim 1, further comprising passing an effluent from the slurry hydrocracking zone to a flash zone.

5. The method according to claim 4, wherein the flash zone comprises a flash drum.

6. The method according to claim 5, further comprising passing a stream from the flash zone to a stripping zone.

7. The method according to claim 6, wherein the stripping zone further comprises a stripper providing the hydrocarbon stream comprising one or more C10-C14 hydrocarbons to the hydroprocessing zone.

8. The method according to claim 1, wherein a vessel pressure is about 1,300-about 140,000 kPa and a vessel temperature is about 250-about 650° C.

9. The method according to claim 1, wherein the internal riser is wholly contained by a shell of the vessel.

10. The method according to claim 9, wherein the hydroprocessing catalyst circulates upwards in the internal riser and drops downward in the shell, and the hydroprocessing catalyst has a mean particle diameter of no more than about 1,000 microns and comprises at least one element of a group 6 and groups 8-10 of the periodic table.

11. The method according to claim 1, further comprising regenerating the hydroprocessing catalyst and returning the hydroprocessing catalyst proximate to a top of the vessel.

12. The method according to claim 1, further comprising providing a slurry hydrocracking catalyst comprising particles having a mean particle diameter of about 2-about 100 microns.

13. The method according to claim 6, further comprising removing at least a portion of a spent catalyst from a stream obtained from the stripping zone.

14. The method according to claim 1, further comprising a fractionation zone for receiving a product stream from the hydroprocessing zone and separating the donor solvent stream provided to the slurry hydrocracking zone.

15. A hydrocarbon conversion method, comprising:
A) providing a hydrocarbon stream comprising one or more C10-C14 hydrocarbons to a hydroprocessing zone comprising a vessel containing an internal riser; wherein a hydroprocessing catalyst circulates within the vessel by at least partially rising within the internal riser;
B) providing a product stream from the hydroprocessing zone to a fractionation zone for obtaining a donor solvent stream; and
C) providing the donor solvent stream at least partially obtained from the hydroprocessing zone to a slurry hydrocracking zone.

16. The method according to claim 15, wherein the fractionation zone further provides a stream comprising one or more C4⁻ hydrocarbons and a stream comprising one or more C5-C9 hydrocarbons.

17. A hydrocarbon conversion apparatus, comprising:
A) a hydroprocessing zone comprising a vessel containing an internal riser, wherein the internal riser is wholly contained by a shell of the vessel; and
B) a slurry hydrocracking zone comprising a slurry hydrocracking reactor in communication with the hydroprocessing zone to receive a hydrocarbon stream at least partially obtained from the hydroprocessing zone.

18. The apparatus according to claim 17, further comprising a fractionation zone adapted to receive a product stream from the hydroprocessing zone and providing the hydrocarbon stream to the slurry hydrocracking zone.

19. The apparatus according to claim 17, further comprising a stripping zone adapted to receive an effluent from the slurry hydrocracking zone and provide one or more C10-C14 hydrocarbons to the hydroprocessing zone.

20. The apparatus according to claim 17, further comprising a regeneration zone adapted to receive a spent catalyst and provide a regenerated catalyst to the hydroprocessing zone.
